(19) United States
(12) Patent Application Publication
(10) Pub. No.: US 2023/0218195 A1
(43) Pub. Date: Jul. 13, 2023

(54) DEVICE AND METHOD FOR CHARACTERIZING PARTICLES OF EXHALED AIR

(71) Applicant: Palas GmbH Partikel- und Lasermesstechnik, Karlsruhe (DE)

(72) Inventors: Dieter KOEHLER, Winkhausen Schmallenberg (DE); Gerhard SCHEUCH, Wohratal (DE); Maximilian WEISS, Weingarten (DE); Frederik WEIS, Herxheim (DE)

(73) Assignee: Palas GmbH Partikel- und Lasermesstechnik, Karlsruhe (DE)

(21) Appl. No.: 18/113,972

(22) Filed: Feb. 24, 2023

Related U.S. Application Data
(63) Continuation of application No. PCT/EP2020/074037, filed on Aug. 27, 2020.

(51) Int. Cl. A61B 5/08; A61B 5/00

(52) U.S. Cl. CPC A61B 5/082; A61B 5/0075

(57) ABSTRACT

A device for characterizing particles of exhaled air. The device comprises an inlet line directed towards an outer environment with a filter for filtering particles. The inlet line is fluidly connected to a breathing line which comprises an interface through which air is breathable. A measurement line is fluidly connected to the breathing line and is fluidly connected to a particle measurement device for determining a parameter corresponding to the particles of the exhaled air. An inventive method comprises the following steps: Directing ex- haled air to a particle measurement device and determining a parameter corresponding to the particles of exhaled air, the parameter being at least one of the following parameters: Particle number, particle concentration (density), particle diameter, particle mass, particle size distribution, particle mass distribution, particle mass concentration, particle number concentration.

This nonprovisional application is a continuation of International Application PCT/EP2020/074037, which was filed on Aug. 27, 2020, and which is herein incorporated by reference.

### BACKGROUND OF THE INVENTION

### Field of the Invention

The invention relates to a device and a method for characterizing particles of exhaled air. The invention further relates to a use of the device, a method of screening a subject for an infectious disease, a method of preventing the spread of an infectious disease, a method of screening a sample of air for an infectious agent, an antiviral agent, an anti-inflammatory agent, a computer program and a computer readable medium.

### Description of the Background Art

In the sense of the invention, particles may refer to particles in a fluid, here exhaled air, which are also known as aerosol particles. An aerosol is a mixture of a gas with solid and/or fluid suspended particles, for example water droplets, soot particles, material abrasion particles, pollen, bacteria, viruses and other organic and chemical substances.

There are known devices and methods for measuring particles of air in general, for example optical photometers. These devices are, however, not sufficiently accurate since they are typically not able to differentiate between the particles contained in the exhaled air and those contained in the surrounding atmosphere since the exhaled air is inevitably mixed with the surrounding air before measurement. Accordingly, these devices are not suitable for any application where a high level of measurement accuracy is requested, like for example in case of diagnostic analysis of human or animal exhaled air.

### SUMMARY OF THE INVENTION

It is therefore an object of the invention to eliminate the disadvantages of the prior art and to provide a device and a method which are capable of delivering more reliable results, particularly where a high level of accuracy is required like for example in diagnostic and medical applications.

In an exemplary embodiment, the object of the invention is solved by a device for characterizing particles of exhaled air which comprises an inlet line directed towards an external environment, wherein the inlet line comprises a filter for filtering particles, wherein the inlet line is fluidly connected to a breathing line which comprises an interface through which air is breathable, wherein the device comprises a measurement line which is fluidly connected to the breathing line and which is fluidly connected to a particle measurement device for determining a parameter corresponding to the particles of the exhaled air.

The object of the invention is also solved by a method for characterizing particles of exhaled air, comprising the following steps: Directing exhaled air to a device for characterizing particles of exhaled air and determining a parameter corresponding to the particles of exhaled air, the parameter being preferably at least one of the following parameters: Particle number, particle concentration, particle diameter, particle mass, particle size distribution, particle mass distribution, particle mass concentration, particle number concentration.

The object of the invention is also solved by a use of the inventive device for characterizing particles in exhaled air. The object of the invention is also solved by a computer program with orders which result in an inventive device executing an inventive method and by a computer readable medium, on which an inventive computer program is saved.

The object of the invention is also solved by a method of screening a subject for an infectious disease, the method comprising the steps of: determining at least one parameter corresponding to particles contained in air exhaled by the subject: Particle number, particle concentration, particle diameter, particle mass, particle size distribution, particle mass distribution, particle mass concentration, particle number concentration; comparing the determined parameter of exhaled particles having a particle diameter within a preselected range to a control parameter of particles of the same diameter range exhaled by a healthy subject; identifying the subject as being a high emitter user, preferably as having at least potentially the infectious disease if the determined parameter fulfills a preset condition; and screening the subject thus identified in a second screen to confirm that the subject has the infectious disease.

The object of the invention is also solved by a method of preventing the spread of an infectious disease, the method comprising the steps of: determining at least one parameter corresponding to particles contained in air exhaled by the subject: Particle number, particle concentration, particle diameter, particle mass, particle size distribution, particle mass distribution, particle mass concentration, particle number concentration; comparing the determined parameter of exhaled particles having a particle diameter within a preselected range to a control parameter of particles of the same diameter range exhaled by a healthy subject; and identifying the subject as being a high emitter user, preferably as having at least potentially the infectious disease if the subject’s determined parameter fulfills at least a preset condition; and isolating the subject or instructing the subject to wear a facemask.

The object of the invention is also solved by a method of screening a sample of air for an infectious agent, the method comprising the steps of: determining at least one of the following parameters (p) of particles contained in a sample of air exhaled by a subject: Particle number, particle concentration, particle diameter, particle mass, particle size distribution, particle mass distribution, particle mass concentration, particle number concentration; comparing the determined parameter of the sample having a particle diameter within a preselected range to a control parameter of particles of the same diameter range in a sample of air exhaled by a healthy subject; identifying the subject as being infected with an infectious agent if the determined parameter fulfills a preset condition; and optionally screening a further sample of air exhaled by the subject thus identified in a second screen to confirm that the subject has the infectious disease.

The object of the invention is also solved by an antiviral agent selected from remdesivir for use in the treatment of a subject, identified by the above method of screening a sample of air as having COVID-19.

The object of the invention is also solved by an anti-inflammatory agent selected from dexamethasone for use in the treatment of a subject identified by the above method of screening a sample of air as having COVID-19.

The invention is based on the idea that exhaled air in general comprises only a part of air available in an environment. In order to reliably characterize exhaled air, the exhaled air needs to be directed towards a particle measurement device. This can for example be achieved by a filter to ensure that only exhaled air is directed to the particle measurement device.

Moreover, determining at least one of the mentioned parameters enables a reliable characterization of exhaled air. The particle number refers to an amount of particles being present in the exhaled air or at least a part thereof. The particle concentration is sometimes also referred to as particle density and refers to the amount of particles per volume, for example per liter air. The particle size distribution refers to the concentration of particles of the aerosol, here exhaled air, as a function of the size of the particles, here their diameter, and provides information on how often which particle sizes are present in the exhaled air. Similarly, the particle mass distribution refers to the concentration of particle masses of the aerosol as a function of the particle diameter. The particle mass concentration refers to the general concentration regardless of the particle diameter. In the sense of the invention, the determining of the at least one parameter can also comprise additional parameters, for example determining particularly local minimum and/or maximum values, or combining the parameter with a weighting function, for example determining a particle size distribution which is weighted by respective particle masses. The mentioned parameters can also be determined for only a part of the particles of the exhaled air. In the sense of the invention, the determining of the parameter also comprises determining the parameter for a preset time interval, which can be user-defined, and preferably calculating the average value for the parameter which may also be weighted by additional parameters as already mentioned. Moreover, the determining of the parameter can also comprise an interpolation of discrete measuring points.

The inlet line, the breathing line and/or the measurement line can comprise a pipe, a pipe socket or a channel through which air can be directed. The filter can comprise a depth filter which is also known as a high efficiency particulate air (HEPA) filter. The filter preferably comprises a filtering efficiency of at least 99.97% of particles having a diameter of 0.3 µm with the efficiency being preferably higher for particles having a smaller and/or greater diameters than 0.3 µm. In the sense of the invention, the particle size is approximated by the diameter of the particles, even if the particles may not form geometrically exact spheres. Preferably, the filter is removable and/or replaceable in order to meet hygienic standards. The filter particularly is form-fittingly connected to the inlet line, for example the filter can be screwed to the inlet line. The connection between the filter and the inlet line is preferably fluidly tight.

In order to create an efficient air flow, the breathing line can be arranged parallel to the inlet line, in particular coaxially to the inlet line. The interface can be configured to be a user interface which preferably comprises a mouthpiece through which a user can breathe air. The interface preferably comprises a mouth-nose-piece or a face mask in order to increase the flow of exhaled air through the device. The interface can be removable, in particular replaceable in order to meet hygienic standards. To this end, the interface can be disposable and/or disinfectable. The interface is preferably fluidly sealable such as to block air flow through the interface in order to improve the purity of the air inside the device.

The measurement line can be arranged perpendicularly to the inlet line and/or to the breathing line to provide an efficient air flow. The device, especially the particle measurement device, preferably comprises at least one air flow generator which is in particular configured to create an air flow with a preset flow rate to and/or inside the particle measurement device, wherein the flow rate is preferably in the range from 0.1 I/min to 101 I/min, particularly in the range from 0.1 I/min to 20 I/min, especially in the range from 1 I/min to 10 I/min. The air flow generator can comprise a suction device, a pump and/or a fan. Preferably, the measurement line and/or the particle measurement device comprise an air flow generator.

The measurement line and/or the particle measurement device can comprise at least one heater which is in particular configured to keep the temperature at a preset value which is preferably in the range from 30° C. to 90° C., especially in the range from 40° C. to 80° C., particularly in the range from 50° C. to 70° C. Condensated droplets of water as particles of the exhaled air can be evaporated so that they do not interfere with the particle measurements. The measurement line, particularly its jacket area, and/or the particle measurement device preferably comprise at least one antistatic and/or electrically conductive component, for example a metal and/or a conductive polymer tubing, so as not to disturb the flow of particles inside the device.

The measurement line and/or the particle measurement device can comprise at least one check valve in order to regulate the flow of exhaled air and to avoid contamination. The diameter of the measurement line is preferably smaller than the diameter of the inlet line and/or the diameter of the breathing line to regulate the air flow to the particle measurement device. The breathing line and the measurement line can be formed integrally, as one piece, preferably as a T-shaped component.

The inlet line, the breathing line and the measurement can comprise, at least sectionally, a measurement chamber, wherein the volume of the measurement chamber is preferably at most 25 ml. The measurement chamber can be arranged between the interface and the filter, preferably to be in fluid connection to the interface and the filter. The measurement chamber can be arranged in fluid connection to the inlet line, the breathing line and the measurement line.

The particle measurement device is capable of determining at least one of the following parameters of the particles of the exhaled air: Particle number, particle concentration, particle diameter, particle mass, particle size distribution, particle mass distribution. The particle measurement device is preferably capable of determining a particle concentration in the range from 0 to 10⁷ particles per liter air, especially in the range from 0.01 to 10⁷ particles per liter air, preferably in the range from 0.01 to 5*10⁶ particles per liter air, particularly in the range from 0.01 to 10⁶ particles per liter air. The particle measurement device can be capable of determining particle diameters in the range from 0.1 µm to 5 µm, especially from 0.1 µm to 1 µm, preferably from 0.2 µm to 5 µm, particularly from 0.3 µm to 5 µm, especially from 0.5 µm to 5 µm.

In order to characterize the particles of exhaled air, the particle measurement device can comprise at least one source for emitting waves, for example electromagnetic and/or acoustic waves. The particle measurement device is preferably an optical particle measurement device which comprises at least one light source. The particle measurement device can comprise additionally a photomultiplier, a photodiode and/or a photometer. In a preferred embodiment of the invention, the light source is capable of emitting polychromatic light and/or light with a least one wavelength in the range from 380 nm to 490 nm. In another preferred embodiment of the invention, the light source is capable of emitting coherent light and can comprise at least one laser element. The light source can comprise at least one LED and/or an optical particle counter which can be provided in the form of an optoelectric sensor.

The particle measurement device can comprise an aerosol spectrometer. Preferably, particles of exhaled air are arranged inside a measuring cell of the aerosol spectrometer in such a way as to the particles can be illuminated by a light beam, wherein scattering light of the particles can be received by a sensor and scattering light signals of the particles can be registered by intensity spectroscopically in such a way that a size distribution of the scattering light signals can be determined which represents a particle size distribution. The direction of movement of the particles inside the measuring cell, the direction of the light beam inside the measuring cell and the direction of the scattering light are arranged perpendicular to one another, respectively. The particle measurement preferably comprises between 1 and 256 channels, particularly between 4 to 256 channels, preferably at least 4 to 256 spectral channels which in particular are capable of detecting light, especially scattering light.

In an example of the inventive method, the method is executed by an inventive device for characterizing particles of exhaled air. A volume of exhaled air of at least 500 ml can be directed to the device, especially to the particle measurement device. The exhaled air can be directed to the device at a preset flow rate which is particularly in the range from 0.1 I/min to 20 I/min, preferably from 1 to 10 I/min. The parameter can be determined for a preset time interval after which a decision parameter is determined, wherein the decision parameter can be a statistical parameter, for example as a preferably weighted average value of the determined parameter. The decision parameter can be compared to a preset value and, depending on the outcome of the comparison, a signal is output.

A cleaning phase can be executed before the determining of the parameter in order to improve the characterization. The cleaning phase can comprise the following steps: Determining the parameter corresponding to the particles of exhaled air for a preset time interval, determining a change parameter based on the parameter and, preferably, outputting a signal if the change parameter fulfills a preset comparison. As one example, the change parameter can be compared to a preset threshold value and a signal can be output if the change parameter is below or above the threshold value.

A sealing checking phase can be executed before the determining of the parameter, preferably before the cleaning phase in order to ensure the quality of the measurement. The sealing checking phase can comprise the following steps: Blocking the flow of exhaled air to the device, directing filtered air of the external environment to the device, determining the above-mentioned parameter for a preset time interval and determining a change parameter based on the parameter and preferably outputting a signal if the change parameter fulfills a preset comparison. The method is particularly only continued if the change parameter fulfills the preset comparison, otherwise the method can be aborted.

For the inventive use, the characterizing can include determining at least one of the following parameters: Particle number, particle concentration, particle diameter, particle mass, particle size distribution, particle mass distribution.

In an example of screening, the second screen can comprise a PCR-based test for detecting the presence of an infectious agent in the subject.

For the inventive method of screening a subject for an infectious disease, the second screen may comprise a PCR-based test for detecting the presence of an infectious agent in the subject.

The inventive method of preventing the spread of an infectious disease may comprise a further step of treating the subject with a therapeutically effective amount of an agent to treat the infectious disease.

Both the inventive methods of screening a subject for an infectious disease and of preventing the spread of an infectious disease are preferably performed using the device described above. In another embodiment, the infectious disease is a viral infection of the lower respiratory tract. In still another embodiment, the infectious disease is COVID-19 and the agent is an anti-viral agent, an immunosuppressive agent, or an antiinflammatory agent. According to another example, the anti-viral agent may be remdesivir and, still in another, the antiinflammatory agent may be a corticosteroid, optionally selected from dexamethasone.

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 is an example of the inventive device in a schematic view,

FIG. 2 is a detailed view of a particle measurement device of the example according to FIG. 1,

FIG. 3 is a flow chart of an example of the inventive method,

FIG. 4 is a flow chart of a sealing checking phase of the inventive method,

FIG. 5 is an exemplary result of the sealing checking phase according to FIG. 4,

FIG. 6 is an exemplary result of a cleaning phase of the inventive method,

FIG. 7 is a flow chart of the cleaning phase according to FIG. 6,

FIG. 8 is an exemplary result of a measurement phase of the inventive method,

FIG. 9 is a flow chart of the measurement phase according to FIG. 8,

FIG. 10 is an exemplary result of a particle concentration determination of exhaled air by a healthy user,

FIG. 11 is an exemplary result of a particle concentration determination of exhaled air by a high emitting user,

FIG. 12 is an exemplary result of a particle size distribution determination of exhaled air by a healthy user,

FIG. 13 is an exemplary result of a particle size distribution determination of exhaled air by a high emitting user, and

FIG. 14 is the inventive device according to an example in a schematic view.

### DETAILED DESCRIPTION

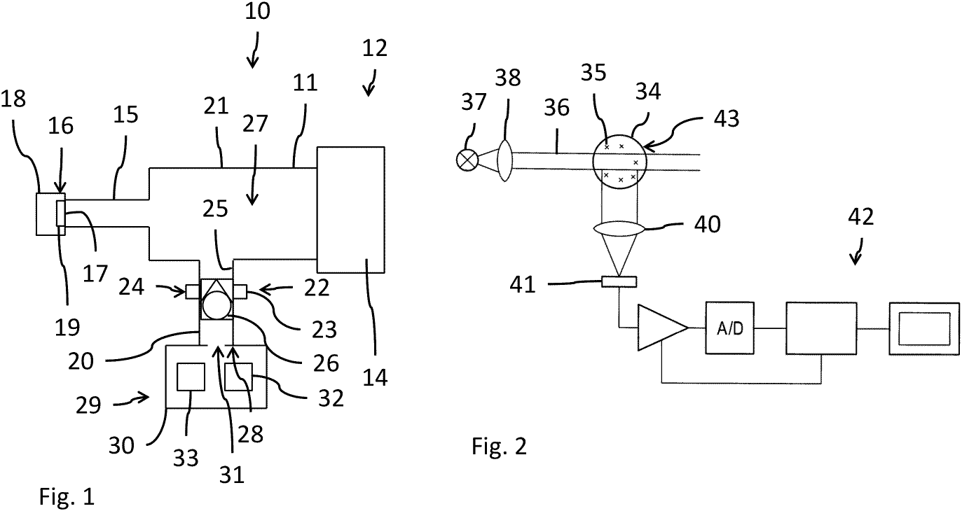

FIG. 1 shows an inventive device 10 for calculating the particle concentration cₙ of exhaled air as a parameter p for characterizing the particles of exhaled air according to a first embodiment in a schematic view. The device 10 comprises an inlet line 11 which is directed to an external environment 12, usually a room in which a user is located. The inlet line 11 is fluidly connected to the external environment 12.

The inlet line 11 comprises a filter 13 which in this embodiment is realized as a depth filter 14 having a porous filtration medium for retaining particles throughout the medium. In this example, the porous medium comprises mats of randomly arranged glass fibers which are not shown in FIG. 1. This type of filters 14 is also known as HEPA filter which filters out at least 99.97% of particles of the air passing through the filter 14. In this embodiment, the depth filter 14 is form-fittingly connected to the inlet line 11 by being screwed to the inlet line 11. The depth filter 14 is replaceable.

The inlet line 11 is fluidly connected to a breathing line 15 which is arranged parallel, especially coaxial to the inlet line 11. The breathing line 15 comprises an interface 16 which is connected to the end face 17 of the breathing line 15 which is directed away from the inlet line 11. The interface 16 in this embodiment is a facemask 18 covering the mouth and nose of a user. Alternatively, a closeable mouthpiece can be used as an interface 16, wherein the nose of the user is sealed by a nasal clamp (not shown). The facemask 18 is replaceable, disposable after being used and disinfectable. By means of a valve 19 inside the facemask 18, the facemask 18 is closeable, especially sealable, in order to prevent air flow between the breathing line 15 and the facemask 18. The diameter of the breathing line 15 is smaller than the diameter of the inlet line 11.

A measurement line 20 is arranged between the inlet line 11 and the breathing line 15 and perpendicular to both of said lines 11, 15. The diameter of the measurement line 20 is smaller than the diameters of both the inlet line 11 and the breathing line 15. The inlet line 11, the breathing line 15 and the measurement line 20 are integrally formed as a T-shaped component 21 wherein the inlet line 11, the breathing line 15 and the measurement line 20 are designed as pipe sockets of the component 21.

The measurement line 20 comprises a heater 24 for keeping the measurement line 20, particularly its inner wall 25, at a preset temperature T of 60° C. The measurement line 20 is made of metal and/or an (electrically) conductive polymer tubing both of which have antistatic properties. The measurement line 20 further comprises a check valve 26 which prevents a return flow of air. The inlet line 11, the breathing line 15 and the measurement line 20 sectionally comprise a measurement chamber 27 with a volume of 25 ml, wherein the measurement chamber 27 is fluidly connected to the depth filter 14 and the facemask 18.

At its end face 28 facing away from the measurement chamber 27, the measurement line 20 is removably connected to a particle measurement device 29 which is capable of characterizing particles 35 of exhaled air. In this embodiment, the particle measurement device 29 is an aerosol spectrometer 30 with part of its design being schematically shown in FIG. 2. The aerosol spectrometer 30 comprises an air flow generator 22, here a fan 23 and/or a pump 33, for generating a defined air flow in the measurement line 20 with a flow rate q_{fl} of a preset value in the range from 0.1 I/min to 101 I/min by which the particles 35 of the exhaled air are directed towards an opening 31 of the aerosol spectrometer 30. Moreover, the aerosol spectrometer comprises a heater 33, as well.

A flow tube 34 of the aerosol spectrometer 30 carrying the particles 35 is shown in FIG. 2 as being arranged perpendicular to the drawing area. The particles 35 in the flow tube 34 are illuminated by a collimated light beam 36 of polychromatic light emitted by a light source 37, here an LED, and a lens 38 with wavelength in the range from 390 nm to 490 nm. By scattering processes, the particles 35 emit scattering light 39 which is perpendicular to both the direction of flight of the particles 35 and the direction of the light beam 36 coming from the LED 37. The scattering light 39 hits a converging lens 40 which focuses the scattering light 39 on an optoeletric sensor 41, comprising here a photomultiplier and a photometer (not shown), which converts the intensity of the scattering light 39 to electric signals. Based on the electric signals, an electronic processing unit 42 determines a particle size distribution cₙ(dₚ) as a function of the particles’ diameters dₚ in order to characterize the particles 35 of the exhaled air. The electronic processing unit also comprises a control module which is electronically connected to the valve 16, the check valve 26, the heater 24, 33, the air flow generator 22, 32 and is capable of executing an inventive procedure which is described below.

The spatial overlap of the light beam 36, the registered scattering light 39 and the registered part of the particles 35 in the flow tube 34 defines a virtual spatial measuring cell 43 in which the particle size distribution cₙ(dₚ) is determined. In the course of the measurement, the light intensity of the scattering light 39 and therefore the hereby determined electrical signal strength is a measure of the size of the particles which is attributed a particle diameter dₚ. The determined particle size distribution cₙ(dₚ) is a function of the particle diameter: cₙ = f(dₚ). The particle size distribution cₙ(dₚ) is determined for discrete particle diameters dₚ as measuring points wherein usually 256 measuring channels are used. To improve the measurement quality, the particle size distribution cₙ(dₚ) are interpolated, preferably by means of cubic splines. The particle concentration cₙ is the sum of the particle size distribution cₙ(dₚ) over every particle diameter dₚ.

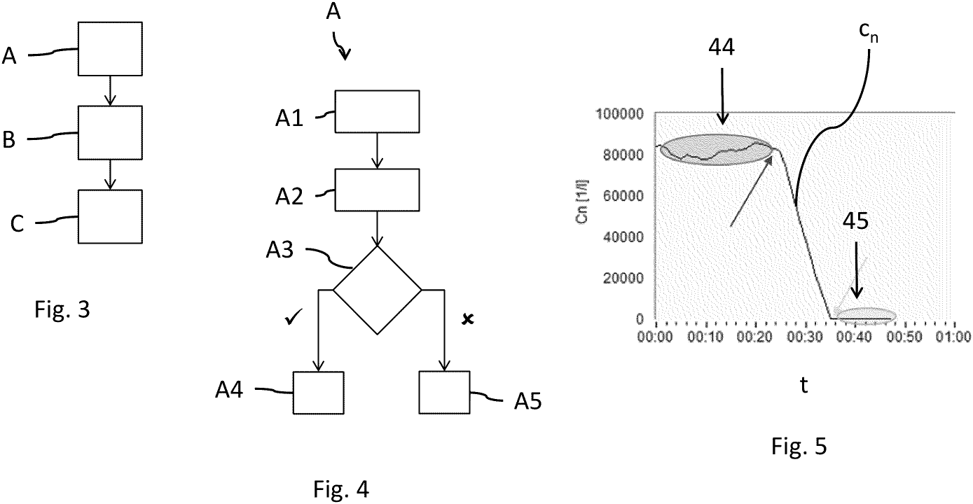

In an embodiment of the inventive method outlined in the flow chart according to FIG. 3, the procedure comprises three phases, a sealing checking phase A with regard to the correct sealing of the device 10, a cleaning phase B and a determining phase C. In this embodiment, the particle concentration cₙ, also known as the particle density, will be determined. The method is described in detail as follows:

The object of the sealing checking phase A is to ensure that the device 10 is correctly sealed and no unfiltered air of the external environment 12 enters the device 10. This phase also removes any residual airborne particles within the device (including facemask, inlet line, breathing line and measurement line). The sealing checking phase A is outlined in the flow chart according to FIG. 4 and begins with a step A1 of opening the valve 19 of the facemask 18, starting of the measurement and determining the particle concentration cₙ inside the device 10. Unfiltered air can enter the facemask 18 so the particle concentration cₙ is comparatively high. FIG. 5 shows the development of the determined particle concentration cₙ over time t during the course of the sealing checking phase A. In a first area 44, the particle concentration cₙ is about 80.000 particles per liter air or 80.000/l. The facemask 18 is then closed A2 which results in that now only filtered air can enter the device 10 (via the depth filter) and is thus measured. As this kind of air contains only a small amount of particles 35, the particle concentration cₙ decreases continuously until it reaches a smaller level which is shown in a second area 45 in FIG. 5. The particle concentration cₙ decreases from about 80.000 particles per liter air to a value of almost 0 over the course of about 10 seconds. The average value of the particle concentration cₙ is measured over a preset time interval Δt₁, in this case 12 seconds. If the average value of the particle concentration cₙ is below a preset threshold value c_{n;t} of less than 1 particle per liter air, preferably 0 particles per liter air (step A3), a signal is output A4 indicating that the device 10 is correctly sealed and can be used for further measurements. If the particle concentration cₙ remains at a higher level than the threshold value c_{n;t}, the sealing of the device is assumed to be damaged and a corresponding warning signal is output A5 by an output device for example a display and/or a speaker.

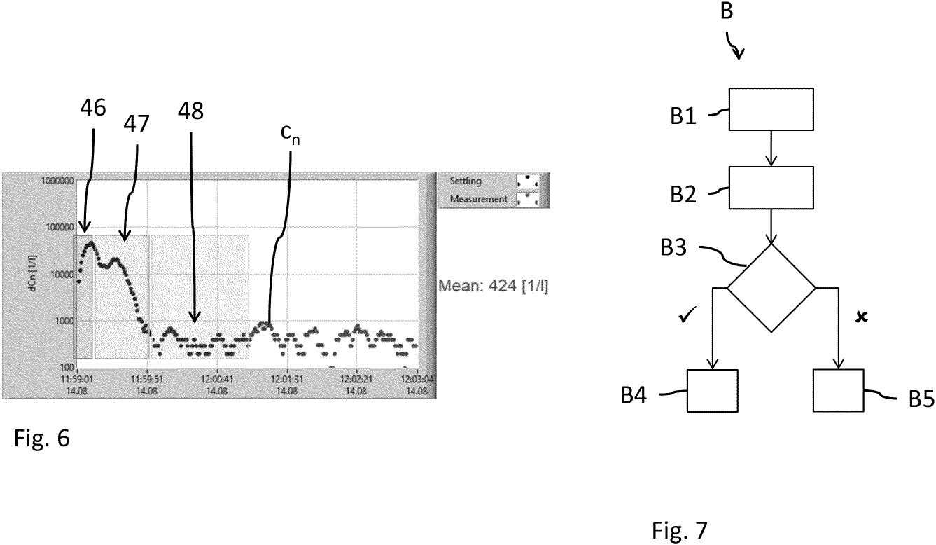

After having verified that the device 10 is properly sealed, the method continues with the cleaning phase B in which the facemask 18 is opened and through which the user breathes. By means of the facemask 18, the exhaled air completely enters the measurement chamber 27 through the measurement line 20 and is subsequently directed towards the aerosol spectrometer 30 where the particle concentration cₙ of the exhaled air is continually measured. As the lungs of the user at first still contain particles from the external environment 12, the device 10 at first registers a still high level of particle concentration cₙ which is shown in a first area 46 of the measurement according to FIG. 6 where the development of the particle density cₙ is shown over time t. It should be noted that the values for the particle concentration cₙ are illustrated on a logarithmic scale so that the value for the particle concentration cₙ in the first area 46 is at its maximum of about 40.000 particles per liter air.

During the course of continued breathing, the user only inhales filtered air through the depth filter 14 and exhales air which is directed B1 into the measurement line 20 so the particle concentration cₙ continually decreases which can be seen in a second area 47 in FIG. 6. This trend continues until the particle concentration cₙ reaches an approximately constant level as shown in a third area 48 of FIG. 6 which corresponds to a state of equilibrium in which the registered particles 35 can be assumed to come only from inside the user’s lungs and air ways, generally the respiratory tract. The value of the particle concentration cₙ in the third area 48 of FIG. 6 is below 1.000 particles per liter air. A change parameter Δcₙ is calculated B2 (for example the variance) and, in a comparison B3, compared to a preset value Δc_{n;th}. If the particle concentration cₙ does not change more than the preset value Δcₙ over a preset time interval Δt₂, here about a minute, the measurement phase C begins and a corresponding signal is output B4. If not, a corresponding warning signal is output B5 indicating that a state of equilibrium has not (yet) been reached. The cleaning phase B illustrated in the flow chart according to FIG. 7.

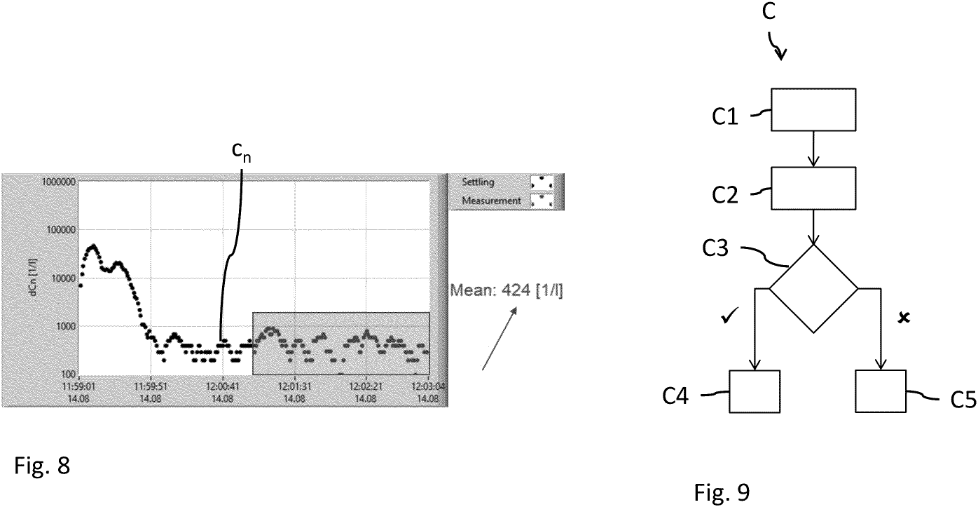

The device 10, more exactly the control module, afterwards executes the measurement phase C in which the particle concentration cₙ is determined C1 over a preset time interval Δt₃, here about two minutes, after which an average value ĉₙ for the particle concentration cₙ as a decision parameter p_{dec} for characterizing the exhaled air is calculated C2. FIG. 8 shows an exemplary measurement, in which the average value ĉₙ for the particle concentration cₙ is calculated to 424 particles per liter air. This determined decision parameter p_{dec} is then compared C3 to a preset value pₕ which is an average value for the particle concentration cₙ of a healthy user. If the decision parameter p_{dec} of the user is higher than the preset value pₕ, the system assumes he user to be a high emitter, also known as “superspreader” (emitting more-than-average amount of particles per liter air) which in some cases indicates a potentially heightened risk of infection and and outputs C5 a corresponding warning signal via the output. If not, the user is considered healthy and a corresponding signal is output C4. FIG. 9 illustrates the measurement phase C by a flow chart.

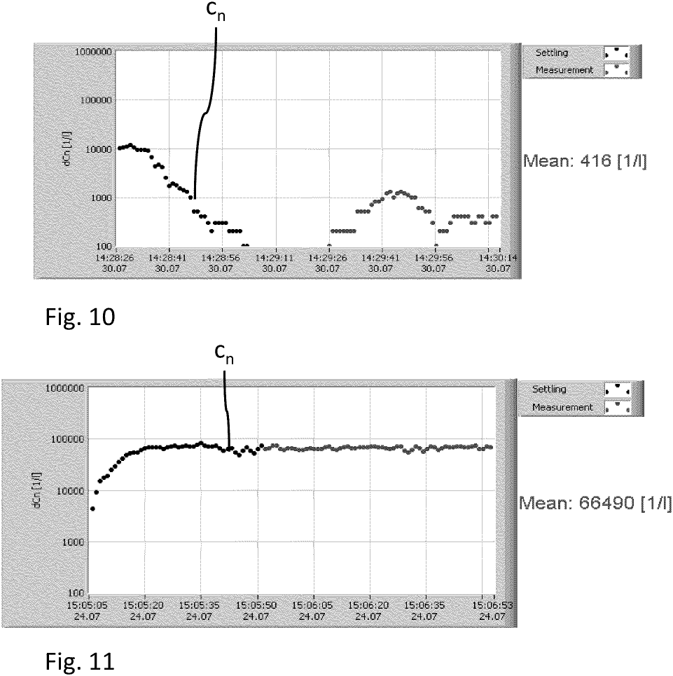

FIG. 10 shows the determining of the particle concentration cₙ for a healthy user with an average value ĉₙ for the particle concentration cₙ calculated to 416 particles per liter air which roughly corresponds to the measurement according to FIG. 8. In contrast, FIG. 11 shows the determined particle concentration cₙ for a high emitting user who might be infectious. It can be noticed that the particle concentration cₙ does not decrease during the time of measurement which suggests that the amount of particles from the user’s lungs is at least as high as particle concentration cₙ of the external environment 12. Correspondingly, the average value ĉₙ for the particle concentration cₙ was calculated to 66.490 particles per liter air which is significantly higher than the corresponding value for the healthy person according to FIG. 10. Accordingly, the device 10 outputs C5 out a warning signal indicating that the user might be a high emitting user and/or at least potentially infectious. The exhaled concentration depends on the breathing manoeuver of the user, e.g. forced breathing, tidal breathing. Preferably, tidal breathing is measured.

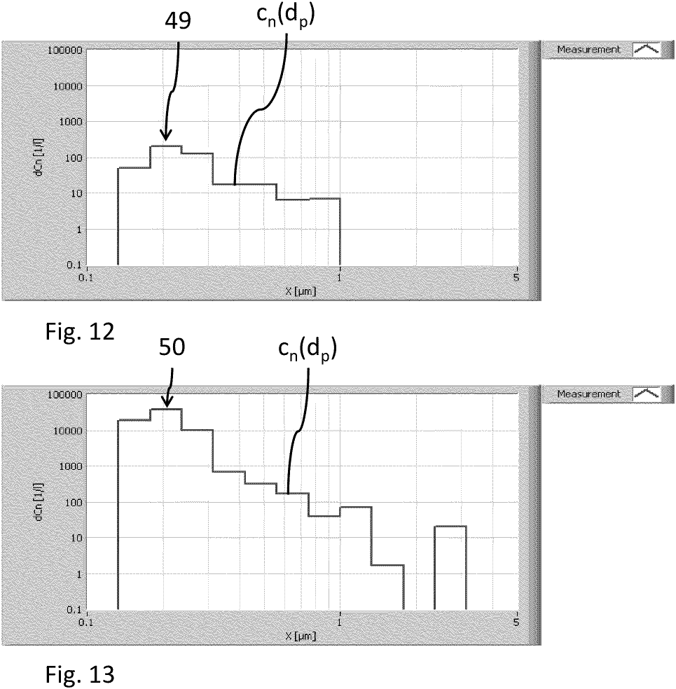

In another example of the inventive method, the particle size distribution cₙ(dₚ) of the exhaled air is additionally determined. FIG. 12 shows the particle size distribution cₙ(dₚ) of a healthy person with both axes being logarithmically displayed. The particle size distribution cₙ(dₚ) is registered by 256 measurement channels of the aerosol spectrometer 30 with each channel representing an interval of particle sizes, here particle diameters dₚ. In this embodiment, the intervals of particle diameters dₚ are logarithmically arranged as can be seen from the x axis in FIG. 12. The y axis corresponds to the particle concentration cₙ(dₚ) for the respective particle diameter dₚ. FIG. 11 shows a global peak 49 of the particle concentration cₙ(dₚ) for a particle diameter dₚ of about 0.2 µm with the peak 49 having a value of about 200 particles per liter air. For particle diameters dₚ greater than 1 µm, no particles are registered. The total particle concentration cₙ can be calculated from the particle size distribution cₙ(dₚ) by integration over the full range of particle diameters dₚ.

FIG. 13 shows a particle size distribution cₙ(dₚ) for a high emitting and/or at least potentially infectious user. The global peak 50 is located at a particle diameter dₚ of about 0.2 µm which was also the case for a healthy user. However, the corresponding particle concentration cₙ(dₚ) for the high emitting and/or at least potentially infectious user is about 30.000 particles per liter air which is significantly higher than the corresponding value of about 200 particles per liter air for a healthy user. Comparing particle concentration values cₙ(dₚ) for specific particle diameters dₚ can be a way of discerning between healthy and high emitting users, especially for particle diameters dₚ in the range from 0.1 µm to 1 µm. The measurement for the high emitting user also shows that particles with diameters dₚ greater than 1 µm are registered with the highest particle diameter dₚ being about 2 µm to 3 µm for which about 11 particles per liter are registered. This was not the case for a healthy user so also the amount of particles cₙ(dₚ) for particle diameters dₚ greater than a preset value can be used to discern a healthy user from a high emitting user.

Looking at FIGS. 12 and 13, the overall particle concentration cₙ is therefore not the only decision parameter p_{dec} on the basis of which a healthy user can be distinguished from a high emitting user. Additional parameters p_{dec} for this means could also be: (Scaled) average particle diameter, shape of particle size distribution, minimum particle diameter, maximum particle diameter, local peaks and/or global peaks. The Inventive method is executed in the form of a computer program which is run on the control module and which is saved on a computer readable medium.

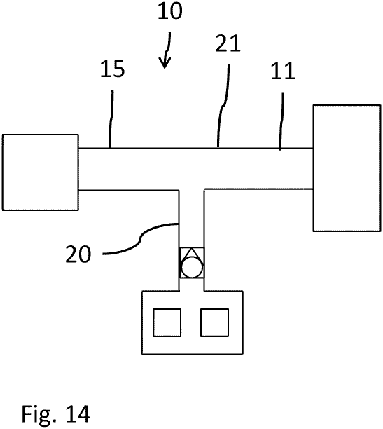

FIG. 14 shows a second embodiment of the inventive device 10 in a schematic view. The inlet line 11, the breathing line 15 and the measurement line 20 are formed integrally as pipe sockets of a single T-shaped component 21 which also comprises the measuring chamber 27. The diameter of the inlet line 11 is equal to the diameter of the breathing line 15, wherein the diameter of the measurement line 20 is smaller than the two former diameters.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A device for characterizing particles of exhaled air, the device comprising:
an inlet line directed towards an external environment, the inlet line comprising a filter for filtering particles, the inlet line being fluidly connected to a breathing line that comprises an interface through which air is breathable; and
a measurement line that is fluidly connected to the breathing line and that is fluidly connected to a particle measurement device to determine a parameter corresponding to the particles of the exhaled air.

2. The device according to claim 1, wherein the breathing line is arranged substantially parallel to the inlet line or arranged coaxially to the inlet line.

3. The device according to claim 1, wherein the interface is fluidly sealable to block air flow through the interface.

4. The device according to claim 1, wherein the particle measurement device comprises an air flow generator configured to create an air flow with a preset flow rate, and wherein a flow rate is in a range from 0.1 I/min to 101 I/min, from 0.1 I/min to 20 I/min, or in the range from 1 I/min to 10 I/min.

5. The device according to claim 1, wherein the measurement line and/or the particle measurement device comprise a heater configured to keep the temperature at a preset value.

6. The device according to claim 1, wherein the measurement line or an inner wall of the measurement line and/or the particle measurement device comprise at least one antistatic and/or electrically conductive component.

7. The device according to claim 1, wherein the measurement line and/or the particle measurement device comprise at least one check valve.

8. The device according to claim 1, wherein a diameter of the measurement line is smaller than a diameter of the inlet line and/or a diameter of the breathing line.

9. The device according to claim 1, wherein the particle measurement device determines at least one of the following parameters of the particles of the exhaled air: particle number, particle concentration, particle diameter, particle mass, particle size distribution, particle mass distribution, particle mass concentration, and/or particle number concentration.

10. The device according to claim 9, wherein the particle measurement device determines a particle concentration in a range from 0 to 10⁷ particles per liter air, in a range from 0.01 to 10⁷ particles per liter air, in a range from 0.01 to 5*10⁶ particles per liter air, or in a range from 0.01 to 10⁶ particles per liter air.

11. The device according to claim 9, wherein the particle measurement device determines particle diameters in a range from 0.1 µm to 5 µm, in a range from 0.1 µm to 1 µm, in a range from 0.2 µm to 5 µm, in a range from 0.3 µm to 5 µm, or in a range from 0.5 µm to 5 µm.

12. The device according to claim 1, wherein the particle measurement device is an optical particle measurement device which comprises at least one light source, wherein the light source is adapted to emit polychromatic light and/or light with at least one wavelength in a range from 380 nm to 490 nm.

13. The device according to claim 12, wherein the particle measurement device comprises an aerosol spectrometer, wherein particles of exhaled air are arranged inside a measuring cell of the aerosol spectrometer to illuminate the particles by a light beam, wherein scattering light of the particles is received by a sensor and scattering light signals of the particles are registered by intensity spectroscopically such that a size distribution of the scattering light signals is determined to represent a particle size distribution.

14. The device according to claim 13, wherein a direction of movement of the particles inside the measuring cell, a direction of the light beam inside the measuring cell and a direction of the scattering light are arranged substantially perpendicular to one another, respectively.

15. The device according to claim 1, wherein the particle measurement device comprises between 1 and 256 channels, between 4 and 256 channels, or at least 4 to 256 spectral channels which are adapted to detect light.

16. A method for characterizing particles of exhaled air, the method comprising:
directing exhaled air to a device for characterizing particles of exhaled air; and
determining a parameter corresponding to the particles of exhaled air, the parameter being at least one of the following parameters:
particle number;
particle concentration;
particle diameter;
particle mass;
particle size distribution;
particle mass distribution;
particle mass concentration; and/or
particle number concentration.

17. The method according to claim 16, wherein the method is executed by a device for characterizing particles of exhaled air, the device comprising:
an inlet line directed towards an external environment, the inlet line comprising a filter for filtering particles, the inlet line being fluidly connected to a breathing line that comprises an interface through which air is breathable; and
a measurement line that is fluidly connected to the breathing line and that is fluidly connected to a particle measurement device to determine a parameter corresponding to the particles of the exhaled air.

18. The method according to claim 16, wherein the exhaled air is directed towards the device at a preset flow rate which is in a range from 0.1 I/min to 101 I/min, in a range from 0.1 I/min to 20 I/min, or in a range from 1 to 10 I/min.

19. The method according to claim 16, wherein the parameter is determined for a preset time interval after which a decision parameter is determined.

20. The method according to claim 19, wherein the decision parameter is compared to a preset value and, depending on the outcome of the comparison, a signal is output.

21. The method according to claim 16, wherein a cleaning phase is executed before the determining of the parameter, wherein the cleaning phase comprises:
determining the parameter corresponding to the particles of exhaled air for a preset time interval;
determining a change parameter based on the parameter; and
outputting a signal if the change parameter fulfills a preset comparison.

22. The method according to claim 16, wherein a sealing checking phase is executed before the determining of the parameter or before a cleaning phase, and wherein the sealing checking phase comprises:
blocking the flow of exhaled air to the device;
directing filtered air of the external environment to the device;
determining the parameter for a preset time interval;
determining a parameter based on the parameter; and
outputting a signal if the parameter fulfills a preset condition.
